(19) United States
(12) Patent Application Publication
(10) Pub. No.: US 2021/0177568 A1
(43) Pub. Date: Jun. 17, 2021

(54) Medical Fabric

(71) Applicant: Asahi Kasei Kabushiki Kaisha, Tokyo (JP)

(72) Inventors: Tokio Okuno, Tokyo (JP); Ryo Fukuda, Tokyo (JP)

(73) Assignee: Asahi Kasei Kabushiki Kaisha, Tokyo (JP)

(21) Appl. No.: 16/759,877

(22) PCT Filed: Nov. 7, 2018
(86) PCT No.: PCT/JP2018/041389

§ 371 Date: Apr. 28, 2020

(30) Foreign Application Priority Data
Nov. 10, 2017 (JP) 2017-217772

(51) Int. Cl. A61F 2/07; D03D 3/02; D03D 15/47; D03D 15/283

(52) U.S. Cl. CPC A61F 2/07; D03D 3/02; A61F 2002/075; D03D 15/283; D10B 2509/06; D03D 15/47

(57) ABSTRACT

Provided is a seamless, cylindrical, high-density medical fabric which is thin and has high strength and low water permeability, the diameter of which can be reduced, which has high sewing strength in a region of at least 10 mm, in the lengthwise direction, from one end thereof, and to which damage can be minimized. The high-density medical fabric according to the present invention satisfies that: (1) both warps and wefts are synthetic fiber multifilament yarns having a total fineness of 60 dtex or lower; (2) the warps each have a single fiber fineness of 0.5 dtex or lower; (3) the cylindrical fabric has a two-wefts insertion woven structure in a region of at least 10 mm, in the lengthwise direction, from one end of the fabric; (4) the fabric has a cover factor of 1600-2400; and (5) the thickness of the fabric is 110 μm or less.

### FIELD

The present invention relates to a medical high density woven fabric. More specifically, the present invention relates to a seamless tubular medical high density woven fabric which has low thickness, high strength, and low water permeability, which enables reduction of the diameter, which has a high suture strength in a region of at least 10 mm in the longitudinal direction, and which is therefore capable of minimizing breakage at the suture site; and a stent graft prepared by suturing and fixing a metal stent to the inner face and/or outer face of the woven fabric using a suture thread, to use the woven fabric as a graft.

### BACKGROUND

Thanks to the recent progress of medical technologies, the therapeutic method for aortic aneurysms is being rapidly replaced from artificial blood vessel replacement to stent grafting, which is less invasive. Conventional artificial blood vessel replacement requires an extensive surgical operation involving thoracotomy or laparotomy, which imposes a heavy burden on the patient. Therefore, its application to elderly patients and patients with comorbidities is limited, and furthermore, it requires long-term hospitalization and hence imposes a heavy economic burden on the patient and the medical facility, which is problematic. In contrast, application of the stent graft operation has been rapidly increasing in recent years since transcatheter endovascular treatment (a therapeutic method in which a thin catheter containing a stent graft compressively inserted therein is introduced through the artery at the base of a leg, which stent graft is then opened and fixed at the site of the aneurysm to block blood flow into the aneurysm, to thereby prevent the aneurysm from rupturing) using a stent graft, which contains a graft such as a medical woven fabric or membrane having a tubular shape combined with a stent that plays a role in maintaining the tubular shape with a metal, does not involve thoracotomy or laparotomy, and therefore the physical and economic burdens can be reduced.

However, as described in PTL 1, the current stent grafts use a stent having a large metal wire diameter and a graft with high thickness, and hence cannot be folded into a small diameter. Thus, they always have a large catheter diameter, and are often not applicable to females and Asians such as Japanese having thin arteries. Thinning of a stent graft requires modification of the shape of the metal stent, the metal wire diameter, and/or the like. However, since fixation of a stent graft to an affected area is basically based on a method in which the stent graft is pressed against the vascular wall utilizing the expansive force of the metal, there is a limitation in the improvement in cases where it affects the expansive force, such as in cases where the stent wire diameter is reduced. On the other hand, thinning of the graft, which occupies a large part of the volume of the stent graft, has also been demanded. However, in cases where, for example, the thickness of an e-PTFE membrane is reduced, the membrane may be stretched to become thinner with time due to the expansive force applied by the stent and the blood pressure, leading to bursting. In view of this, PTL 1 proposes use of a superfine polyester fiber having both high biological safety and moldability.

As described in PTL 2, in cases where the graft is made of a woven fabric composed of fibers, or made of a knitted fabric, the thinning causes blood leakage from the graft itself, which prevents the therapeutic effect. In particular, branched stent grafts used for the treatment of abdominal aortic aneurysms tend to cause leakage from the boundary portion at which the aorta is branched to the lower limbs (left and right iliac arteries), and this problem becomes more obvious as the thickness decreases. Moreover, the branched portion (boundary portion) tends to receive an extension or bending stress, which may cause rupture of a membrane-type graft. In woven fabric-type grafts, the boundary portion is hand-sewn, or the end face treatment is carried out using a thermal cutter, to prevent blood leakage or rupture at the boundary portion site. However, these countermeasures are still insufficient. In view of this, for achieving both the prevention of leakage from the branched portion (boundary portion) and the reduction of the diameter, PTL 2 proposes a seamless tubular medical high density woven fabric using a polyester multifilament yarn having a monofilament fineness of not more than 0.5 dtex as the weft yarn, wherein the woven texture of the branched portion (boundary portion) is constituted by a single texture.

Since the seamless tubular medical high density woven fabrics according to PTL 1 and/or 2 employ a polyester multifilament yarn having a monofilament fineness of not more than 0.5 dtex as the weft yarn, they can reduce the thickness of the graft, and can achieve reduction of the diameter of the stent graft while maintaining the required low water permeability, high burst strength, and thinness. However, in cases of their use for a stent graft in which a metal stent is sutured and fixed to the inner face and/or outer face of the woven fabric using a suture thread, a sufficient suture strength cannot be maintained because of low tensile strength of the superfine fiber, so that breakage at the suture site or the like may occur after the placement in the body, to cause leakage, intratubular obstruction due to folding of the graft, leakage into the aneurysm (endoleak), or the like.

### CITATION LIST

### Patent Literature

[PTL 1] WO 2013/137263

[PTL 2] Japanese Unexamined Patent Publication (Kokai) No. 2016-123764

### SUMMARY

### Technical Problem

In view of the problems in the conventional techniques, an object of the present invention is to provide a seamless tubular medical high density woven fabric which has low thickness, high strength, and low water permeability, which enables reduction of the diameter, which has high suture strength in a region of at least 10 mm in the longitudinal direction, and which is capable of minimizing breakage.

### Solution to Problem

As a result of intensive study and experiments, the present inventors discovered that, by using a synthetic polyester multifilament fiber having a monofilament fineness of not more than 0.5 dtex as weft yarn to provide a tubular woven fabric including a two-weft insertion woven structure in a region of at least 10 mm in the longitudinal direction, a decrease in the suture strength can be prevented in cases where a metal stent is sutured and fixed using a suture thread, thereby completing the present invention.

More specifically, the present invention is as follows.

[1] A seamless tubular medical high density woven fabric, satisfying the following requirements (1) to (8):

(1) both warp yarn and weft yarn are synthetic multifilament fibers having a total fineness of not more than 60 dtex;

(2) the weft yarn has a monofilament fineness of not more than 0.5 dtex;

(3) the tubular woven fabric includes a two-weft insertion woven structure in a region of at least 10 mm in the longitudinal direction from one end of the tubular woven fabric;

(4) the woven fabric has a cover factor of 1600 to 2400; and

(5) the woven fabric has a thickness of not more than 110 μm.

[2] The medical high density woven fabric according to [1], wherein the weft yarn is a synthetic polyester multifilament fiber having a monofilament fineness of not more than 0.2 dtex.

### Advantageous Effects of Invention

The seamless tubular medical high density woven fabric according to the present invention is a seamless tubular medical high density woven fabric which has low thickness, high strength, and low water permeability, which enables reduction of the diameter, which has high suture strength in a region of at least 10 mm in the longitudinal direction, and which is capable of minimizing breakage. Therefore, the woven fabric is useful as the graft for a stent graft in which the graft is sutured and fixed to a metal stent using a suture thread.

### BRIEF DESCRIPTION OF DRAWINGS

FIG. 1 is a woven structure for a case where the warp and weft in the front side of a double-woven texture form a plain texture, and illustrates a woven structure designed only for the warp and weft in the front side, and a 3D schematic diagram thereof.

FIG. 2 is a woven structure for a case where the warp and weft in both the front side and the back side of a double-woven texture form a plain texture, and illustrates a woven structure designed for the double weaving, and a 3D schematic diagram thereof.

FIG. 3 is a woven structure for a case where the warp and weft in the front side of a double-woven texture form a two-weft insertion woven structure, and illustrates a woven structure designed only for the warp and weft in the front side, and a 3D schematic diagram thereof.

FIG. 4 is a woven structure designed for a case where the warp and weft in both the front side and the back side of a double-woven texture form a two-weft insertion woven structure, and a 3D schematic diagram thereof.

### DESCRIPTION OF EMBODIMENTS

An embodiment of the present invention is described below in detail.

The medical high density woven fabric of the present embodiment is a seamless tubular medical high density woven fabric, satisfying the following requirements (1) to (8):

(1) both warp yarn and weft yarn are synthetic multifilament fibers having a total fineness of not more than 60 dtex;

(2) the weft yarn has a monofilament fineness of not more than 0.5 dtex;

(3) the tubular woven fabric includes a two-weft insertion woven structure in a region of at least 10 mm in the longitudinal direction from one end of the tubular woven fabric;

(4) the woven fabric has a cover factor of 1600 to 2400; and

(5) the woven fabric has a thickness of not more than 110 μm.

Both warp yarn and weft yarn constituting (removed from) the seamless tubular medical high density woven fabric of the present embodiment are synthetic multifilament fibers having a total fineness of not more than 60 dtex. The total fineness is preferably 7 dtex to 60 dtex from the viewpoint of thinness and strength of the woven fabric for a stent graft. In cases where the total fineness is not less than 7 dtex, the woven fabric can have a sufficient strength for its practical use. In cases where the total fineness is not more than 60 dtex, the woven fabric is not too thick, and can meet the demand for reduction of the diameter of the stent graft. From the viewpoint of satisfying both the thinness of the woven fabric and the practical performance, the total fineness is more preferably 10 dtex to 50 dtex, still more preferably 15 dtex to 40 dtex.

The weft yarn constituting (removed from) the woven fabric of the present embodiment is a superfine fiber having a monofilament fineness of not more than 0.5 dtex. In cases where the monofilament fineness is not more than 0.5 dtex, affinity with vascular endothelial cells increases to promote integration of the woven fabric with the vascular wall tissue, so that prevention of movement or detachment of the stent graft in the blood vessel, and suppression of thrombogenesis can be expected. From the viewpoint of thinness and cell affinity of the woven fabric, the fiber has a monofilament fineness of preferably not more than 0.4 dtex, more preferably not more than 0.3 dtex, still more preferably not more than 0.2 dtex. There is no lower limit of the monofilament fineness. From the viewpoint of the processability during the warping, weaving, and the like in the woven fabric production process, and achievement of the burst strength of the woven fabric, the monofilament fineness is preferably not less than 0.01 dtex,

The warp yarn constituting (removed from) the woven fabric of the present embodiment has a monofilament fineness of preferably not less than 1.0 dtex, more preferably not less than 1.3 dtex, still more preferably not less than 1.4 dtex. In cases where the warp yarn has a monofilament fineness of not less than 1.0 dtex, the warp yarn can maintain a higher tensile strength compared to the superfine fiber that is the weft yarn, handling during the weaving can be made easier, and the shape stability as a tubular woven fabric can be improved.

The tubular woven fabric of the present embodiment includes a two-weft insertion woven structure in a region of at least 10 mm in the longitudinal direction.

This woven structure region may be present for not less than 10 mm in the longitudinal direction from one end of the tubular woven fabric. The woven structure region is a region of preferably not less than 10%, more preferably not less than 30% in the longitudinal direction of the tubular woven fabric. There is no upper limit of the woven texture region, and the entire tubular woven fabric (100%) is especially preferably this woven structure. In cases where the woven structure region is present for not less than 10 mm from one end, a sewing width having a sufficient strength for suturing to the stent can be secured at the end. The woven structure region produces a higher strength-maintaining effect especially in cases where the weft yarn is a superfine fiber having a monofilament fineness of not more than 0.5 dtex.

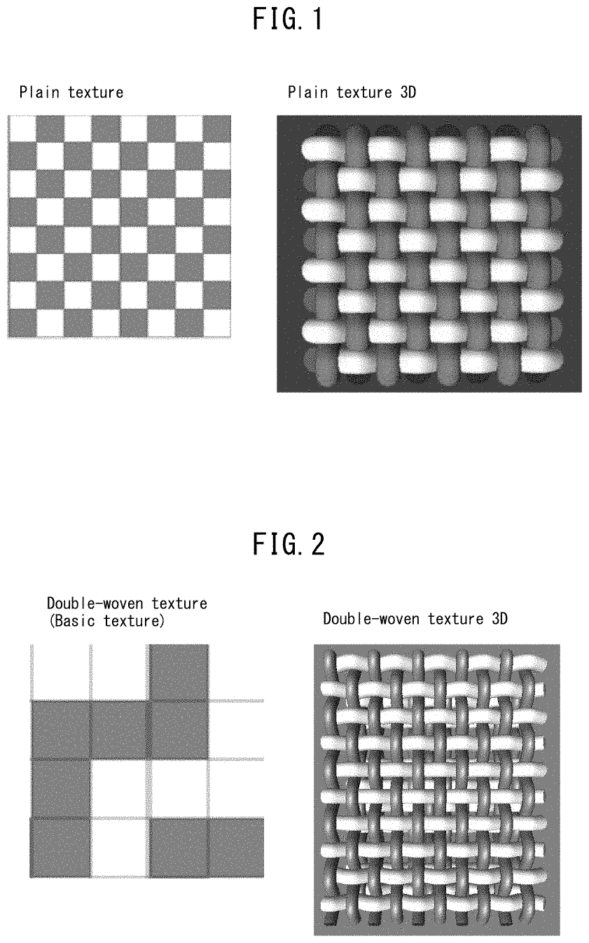

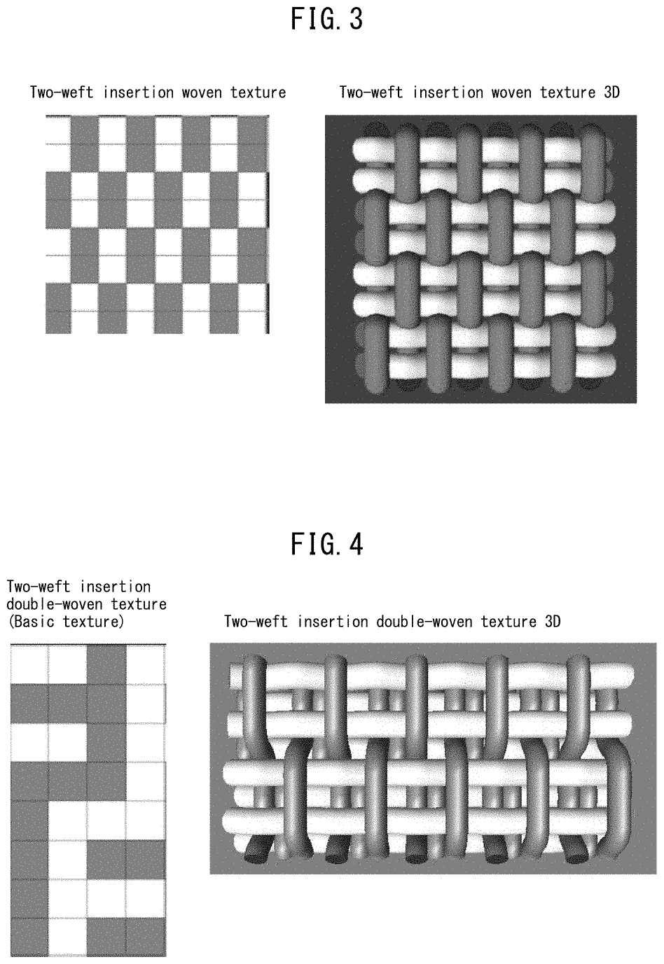

By positioning this region proximally (in the side distant from the leg, opposite to the flow of the blood) in the blood circulatory system where the stent graft is placed, and providing the two-weft insertion woven structure shown in FIG. 3 or 4 as the woven structure in this region, the suture strengths in the warp direction, 45° direction, and weft direction can be increased when a metal stent is sutured and fixed to the inner face and/or outer face of the woven fabric using a suture thread, compared to, for example, the one-weft insertion woven structure (plain double-woven texture) shown in FIG. 1 or 2. Moreover, after the placement in the body, breakage at the suture site, leakage, intratubular obstruction due to folding of the graft, leakage into the aneurysm (endoleak), and the like can be prevented. In cases where the woven structure in the region is the two-weft insertion woven structure shown in FIG. 3 or 4, the suture strength in each of the warp direction, 45° direction, and weft direction of the woven fabric can be not less than 11 N.

The term “one end” as used herein means one of the ends in cases where the tubular woven fabric is a straight fabric having no branch portion, or means the opening of the large-diameter portion in cases where the tubular woven fabric is a branched fabric including a large-diameter portion and a branch portion. Examples of the two-weft insertion woven structure include 2/1 rib, 2/2 twill, 2/2 basket. In cases where 2/1 rib is employed in the present disclosure, the weft yarn is firmly constrained by the warp yarn, and therefore yarn shifting (aperture generation) hardly occurs. Further, because of the presence of the two weft yarns, the strength can be advantageously increased, which is preferred.

The woven fabric of the present embodiment needs to have a cover factor of 1600 to 2400. A cover factor of less than 1600 means that the woven fabric has a low weaving density, which is likely to cause blood leakage from the woven fabric itself. In cases where the cover factor exceeds 2400, the density is high, and blood leakage can be functionally prevented, but there are problems in, for example, that folding becomes difficult due to hardness of the woven fabric itself, and that the woven fabric is not suitable for reduction of the diameter. The cover factor is preferably 1800 to 2300, more preferably 2000 to 2200. The cover factor in the warp direction and the cover factor in the weft direction are preferably, but do not necessarily need to be, almost the same. In cases where the cover factor in the warp direction is higher, the high density woven fabric can be more easily produced.

The cover factor (CF) can be calculated according to the following equation:

CF=(√dw)×Mw+(√df)×Mf

{wherein dw represents the total fineness (dtex) of the warp yarn; Mw represents the weaving density (yarns/2.54 cm) of the warp yarn; df represents the total fineness (dtex) of the weft yarn; and Mf represents the weaving density (yarns/2.54 cm) of the weft yarn}. In the two-weft insertion texture described above, CF is calculated assuming a single yarn having a fineness which is the sum of the finenesses of the two wefts.

The woven fabric of the present embodiment is a seamless tubular woven fabric, that is, a double-woven fabric. For a graft to be used for a stent graft, a sheet-like woven fabric or membrane material may be formed into a tubular shape, and then the ends may be bonded together using an adhesive, or may be sewn together. However, this increases the thickness in the bonded or sewn portion, to prevent compact folding. Thus, a seamless woven fabric is preferred for the reduction of the diameter. Further, because the weft yarn continuously constitutes the woven fabric, bonding and sewing, which are carried out in cases where a flat, non-tubular woven fabric or membrane material is used, and which are laborious manual processes that cause variations, can be eliminated, and moreover, leakage can be reduced. Further, because of the elimination of the surface roughness, smooth flow of blood can be effectively achieved.

Examples of the basic woven structure of double weaving in the woven fabric of the present embodiment include, but are not limited to, the plain weave, twill weave, and satin weave, which may be used individually or in combination. From the viewpoint of thinness and strength of the woven fabric, and reduction of blood leakage, a plain-weave structure is preferred. However, as described above, the tubular woven fabric of the present embodiment needs to include a two-weft insertion woven structure in a region of at least 10 mm in the longitudinal direction from one end of the tubular woven fabric. For any of the woven structures described above, each of the warp density and the weft density of the woven fabric of the present embodiment is preferably not less than 100 yarns/2.54 cm, more preferably not less than 120 yarns/2.54 cm, still more preferably not less than 140 yarns/2.54 cm. Although there is no upper limit, the density is substantially not more than 250 yarns/2.54 cm from the viewpoint of weaving.

The woven fabric of the present embodiment needs to have a thickness of not more than 110 μm. In cases where the thickness is not more than 110 μm, reduction of the diameter upon folding can be achieved, and therefore the woven fabric can be contained in a desired catheter. The thickness is preferably within the range of 10 μm to 90 μm. In this case, the woven fabric can be easily contained in a catheter having a small diameter, and a delivery system can be provided such that the woven fabric can be easily released upon the release in the affected area. In cases where the woven fabric has a thickness of more than 10 μm, a sufficient burst strength can be retained. The thickness of the woven fabric herein is defined as follows. In an area in the circumferential direction and the longitudinal direction (5 cm to 30 cm) of the tubular woven fabric, 10 sites are arbitrary selected, and the thickness at each site is measured using a thickness gauge. The average of the measured values is the thickness of the woven fabric. In the measurement of the thickness of the woven fabric, the thickness variation Z represented by the following equation:

Z (%)=(Zav−Zi)/Zav×100

{wherein Zav represents the average of the 10 measured values; Zi represents the measured value at each point; and i represents an integer of 1 to 10}
at each measurement point is preferably within ±15%.

In cases where the thickness variation is negatively large to exceed −15%, it may be impossible to place the woven fabric in a desired catheter, for example, a catheter with a hole having a diameter of 6 mm, even when the average thickness of the folded woven fabric is not more than 110 μm. Further, portions with a thickness variation of more than 15% have low thickness, and therefore have poor burst strength and poor water penetration-preventing performance. The thickness variation Z is more preferably within ±12%, still more preferably within ±10%.

For example, the thickest blood vessel for which a stent graft is used is the thoracic aorta, which usually has an inner diameter of about 40 to 50 mm. For reduction of the physical burden on the patient, and for application to a wider range of patients, a stent graft with a maximum inner diameter of 50 mm is required to be insertable into an 18-French (6-mm inner diameter) or smaller catheter in the cases of the thoracic aorta. According to the past studies by the present inventors, it became clear that the maximum thickness of a tubular woven fabric having an inner diameter of 50 mm that can pass through a hole having a diameter of 6 mm is 110 μm. Since this thickness does not largely change even in cases where the tubular woven fabric has a different inner diameter, the standard for the thickness of the woven fabric is set to 110 μm or less in the specification of the monofilament fineness and the total fineness of the superfine polyester fiber used in the woven fabric for the stent graft.

The woven fabric of the present embodiment preferably has a burst strength of not less than 100 N. In cases where the woven fabric has a burst strength of not less than 100 N, when the woven fabric is used as a woven fabric for a stent graft, its bursting due to the expansive force of the stent does not occur, which is advantageous from the viewpoint of safety during the use. The burst strength is more preferably not less than 150 N, still more preferably not less than 200 N. There is no upper limit of the burst strength of the woven fabric. From the viewpoint of the balance with the thinness of the woven fabric, the burst strength is substantially not more than 500 N.

The water permeability of the woven fabric itself of the present embodiment is preferably not more than 500 ml/cm²/min. The water permeability of the woven fabric is an index for prevention of blood leakage, and, in cases where the water permeability is not more than 500 ml/cm²/min, blood leakage from the wall surface of the woven fabric can be kept low. The water permeability of the woven fabric is more preferably not more than 300 ml/cm²/min, still more preferably not more than 200 ml/cm²/min.

Usually, by using the woven fabric of the present embodiment as a graft, and sewing the graft to a metal stent using a suture thread, a stent graft is prepared as the final product. In cases where a large needle hole is opened in the woven fabric during this process, blood leakage occurs therefrom. In such cases, the medical woven fabric of the present embodiment preferably has a water permeability of not more than 500 cc/cm²/min after the needle puncture. The water permeability after the needle puncture herein is a value measured after arbitrarily passing a sewing-machine needle (DB×1 normal needle #11, manufactured by Organ Needle Co., Ltd.) 10 times per 1 cm². For reduction of the size of the needle hole, use of a superfine polyester fiber is effective. This is because, when monofilaments in the woven texture are forced to open by the needle, the gaps at the crossing points of the warp yarn and the weft yarn are filled since the monofilaments are flexible, so that the needle hole is less likely to remain, and hence the water permeability after the needle puncture can be kept low.

The tubular woven fabric of the present embodiment may have a straight shape, may include a branch portion, or may include a tapered portion having changing diameters. The branched portion is a portion in which a tubular large-diameter portion is continuously branched into two or more branch portions. For part of the woven texture in the boundary portion between the large-diameter portion and the branch portions, for example, 2/2 basket, 2/2 twill, 2/1 twill, 3/3 basket, or the like may be reasonably used as a texture for the woven structure. The texture may also be a 1/2 rib, 2/1 rib, or plain woven texture. These may be used in combination, and may be selected as long as no problem occurs in terms of weaving or handling.

In cases where the woven fabric of the present embodiment has branch portions, the branch portions may have different diameters. Although the branch portions may have the same length, one branch is generally longer than the other. This is because, for example, in treatment of an abdominal aneurysm, a catheter containing a stent graft having a long branch portion compressively inserted therein is introduced through the iliac artery in one side so that the stent graft is placed in the aneurysm, and then a short straight stent graft is inserted from the other iliac artery to bind it to the above stent graft.

In cases where the woven fabric of the present embodiment has branch portions, for example, during weaving of one branch portion, the warp yarns constituting the other branch portion may be on standby at the upper shed position, or may be on standby at the lower shed position. The woven texture may be produced in an easy-to-weave pattern. There are no particular limitations in cases where the number of warp yarns is small and the load on Jacquard machines or dobby machines is small, such as in cases of a graft base fabric. In cases of weaving of a woven fabric having branch portions, the number of shuttles to be provided is preferably the sum of the number of the branch portions and the number of the large-diameter portion. For example, in cases where two branch portions are woven, three shuttles containing weft yarns are preferably provided. However, since one of the branch portions can be woven with the shuttle used for weaving the large-diameter portion, the weaving is also possible with two shuttles.

In cases where the woven fabric of the present embodiment is a straight fabric having no branch portion, weaving is possible by providing one shuttle containing the weft yarn, and the weft yarn can be continuous. The woven fabric of the present embodiment may be coated with collagen, gelatin, or the like as long as the requirements of the thickness, outer diameter, and the like described above are satisfied.

In the case of two-weft insertion in the woven fabric of the present embodiment, one shuttle in the loom may be used, and the warp yarns may be shed such that the two weft yarns are arranged in the same shed, or two shuttles may be used to insert the two weft yarns in the same shed. Further, in order to make the weft yarns nearly flatly aligned to suppress an increase in the thickness, for example, the shed for two adjacent warp yarns per 20 yarns may be set to 1/1 plain shed to provide a portion where the two warp yarns and the two weft yarns inserted therein form 1/1 plain. These may be appropriately selected as long as the performance is not affected. In any case, there is no need to provide another shuttle containing a weft yarn, and the preparation is possible just by changing the weaving program.

In the region where the two weft yarns are inserted, warp yarns are preferably arranged one by one. In the so-called rip-stop texture, which has a large number of sites where not less than two each of warp yarns and weft yarns are adjacent to each other to form a lattice shape, there are regions having high thickness in the longitudinal direction of the tubular woven fabric, so that the folding diameter cannot be reduced, and problems such as formation of irregularities are likely to occur, which is not preferred.

The woven fabric of the present embodiment is usually used as a stent graft by combination with a stent (spring-shaped metal), which acts as an expandable member. Examples of the type of the stent graft include: a simple straight type, which has a tubular shape; and a branched type and a fenestrated type, which are applicable to branched blood vessels. For the expandable member, a self-expanding material using a shape-memory alloy, superelastic metal, or synthetic polymer material may be used. The expandable member may have any of the designs of the conventional techniques. For example, the woven fabric of the present embodiment may be used as a graft, and a zigzag metal stent may be sutured and fixed to the inner face and/or outer face of the woven fabric using a suture thread. As the expandable member, a member that is expanded by a balloon may be applied instead of the self-expanding material. In a stent graft as a preferred mode of the present invention, the gap between the stent and the graft is preferably not more than 2 mm.

Both the warp yarns and the weft yarns used in the present embodiment are preferably polyester fibers. In particular, the superfine polyester fiber used as the weft yarn preferably has a tensile strength of not less than 3.5 cN/dtex and a tensile elongation of not less than 12%. In cases where the tensile strength of the superfine polyester fiber is not less than 3.5 cN/dtex, the woven fabric for a stent graft can produce excellent mechanical/physical properties. From the viewpoint of stable processability in the weaving process of the woven fabric, the superfine polyester fiber in the present embodiment more preferably has a tensile strength of not less than 3.8 cN/dtex, still more preferably not less than 4.0 cN/dtex. From a similar viewpoint, the superfine polyester fiber in the present embodiment more preferably has a tensile elongation of not less than 15%, still more preferably not less than 20%. Since the superfine polyester fiber has a low monofilament fineness, it tends to generate fluff. Thus, a coating may be formed on the yarn by application of a sizing agent or a lubricant, or ease of handling during the weaving may be improved by improving the bundling property of the yarn by twisting or the like. Such a polyester fiber can be prepared using, for example, a production method described in WO 2103/137263.

In weaving of the woven fabric of the present embodiment, the warp yarn may be subjected to twisting at 50 to 1000 T/m, and the twisted yarn may be further subjected to application of a sizing agent, lubricant, or WAX agent. Even without the twisting, application of a sizing agent, lubricant, or WAX agent is effective for suppressing the fluff during the weaving, to improve the weaving performance. However, from the viewpoint of biological safety, sizing is preferably not carried out, and twisting at 300 to 700 T/m alone is preferably carried out for warping of the warp yarn. Even in this case, the spinning oil agent during the production of the original yarn is adhering to the warp yarn. The weft yarn may also be subjected to further application of a spinning oil agent or another oil agent, or may be subjected to twisting at 50 to 200 T/m, to decrease friction and hence to improve the weaving performance. A method suitable for the weaving may be employed as appropriate.

Examples of materials constituting the woven fabric of the present embodiment, other than the superfine polyester fiber, include polyester fibers other than those described above, polyamide fibers, polyethylene fibers, and polypropylene fibers. These may be either monofilaments or multifilaments, and may be used in combination with one or more fiber materials depending on the purpose. Regarding the mode of the combination, a polyester fiber described above may be twisted with another fiber to provide a composite fiber, or another fiber may be used as, or may be partially used as a part of, the warp yarn or the weft yarn of the woven fabric.

In the superfine polyester fiber, the content of the PET component is preferably not less than 98% by weight, that is, the content of the components other than PET is preferably less than 2% by weight. The components other than PET herein means components incorporated into the molecular chains by copolymerization or the like, and components adhering to the surface of the polyester fiber, such as copolymerized PET, polyamide and polystyrene, and copolymers thereof, sea component polymers used for production of sea-island superfine PET fibers, such as polyethylene and polyvinyl alcohol, and degradation products of the sea component polymers. In the present embodiment, the components other than PET preferably do not include PET-derived monomers or oligomers such as ethylene glycol, terephthalic acid (TPA), monohydroxyethylene terephthalate (MHET), or bis-2-hydroxyethyl terephthalate (BHET). The content of the components other than PET in the superfine polyester fiber is preferably less than 1% by weight, more preferably less than 0.5% by weight, still more preferably 0.

The woven fabric of the present embodiment effectively functions not only as a woven fabric for a stent graft, but also as a material to be implanted in the body, such as an artificial blood vessel, artificial fiber cloth, antiadhesive agent, or artificial valve. Further, the woven fabric effectively functions not only as the material to be implanted in the body, but also as a medical material to be used outside the body, such as a hemofiltration material, cell separation membrane, cell adsorbent, or cell culture substrate.

The woven fabric of the present embodiment uses superfine polyester fibers as at least part of the warp yarns and the weft yarns from the viewpoint of achievement of the strength of the stent graft and prevention of blood leakage. From the viewpoint of thinness of the woven fabric, the woven fabric of the present embodiment needs to contain not less than 20% by weight superfine polyester fibers. In cases where the component ratio of the superfine polyester fibers in the present embodiment in the woven fabric is not less than 20% by weight, the thickness of the woven fabric does not exceed 110 μm, so that reduction of the diameter can be realized. Further, in cases where the component ratio of the superfine polyester fibers is not less than 20% by weight, excellent integration with the stent can be achieved. In the woven fabric of the present embodiment, the component ratio of the superfine polyester fibers is preferably not less than 30% by weight. Although the superfine polyester fibers in the present embodiment may be used for both the warp yarn and the weft yarn of the woven fabric, the superfine polyester fibers are especially preferably used for the weft yarn from the viewpoint of better integration with the stent.

In the method of producing a superfine polyester fiber suitable for use in the woven fabric of the present embodiment, a finishing agent may be applied to a fiber bundle to improve processability during the subsequent warping and weaving processes. As the finishing agent, a mineral oil-derived lubricant, a water-soluble lubricant, or the like is used. The oil application rate of the finishing agent is preferably 0.6% by weight to 3% by weight, more preferably 1.2% by weight to 2.8% by weight, still more preferably 1.5% by weight to 2.5% by weight, from the viewpoint of processability during the bulking process and the weaving/knitting process.

In the method for producing the superfine polyester fiber, tangling treatment is preferably carried out at an undrawn-yarn stage or a drawn-yarn stage from the viewpoint of reducing fluff and yarn breakage during the warping and knitting/weaving processes, and improving the unwinding property. For the tangling treatment, a known tangling nozzle is preferably employed, and the number of tangles is preferably within the range of 1 to 50 tangles/m. The thermal shrinkage stress of the superfine polyester fiber used in the weaving is preferably not less than 0.2 cN/dtex within the temperature range of 80° C. to 200° C., from the viewpoint of securing a thermal shrinkage stress of not less than 0.05 cN/dtex as a superfine polyester fiber constituting the woven fabric of the final product of the stent graft (after sterilization treatment).

In a preferred mode of the present embodiment, the stent graft is delivered through blood vessels in a state where the stent graft is inserted in a catheter. Since the stent graft in the present embodiment uses a woven fabric having a thickness of not more than 90 μm, it is thin and highly flexible. It can therefore be inserted into a small-diameter catheter, and, as a result, the stent graft can be easily delivered through blood vessels with a reduced risk of damaging the vascular wall. As the catheter, those using conventional techniques, such as a tube-type catheter or balloon-type catheter, may be preferably used. The stent graft inserted in a small-diameter catheter in the present embodiment can be delivered through blood vessels and placed therein using a conventional delivery system. In cases where the tubular seamless woven fabric of the present embodiment is used as a woven fabric for a stent graft, the diameter of the stent graft can be reduced, so that the physical and economic burdens on the patient can be reduced by, for example, shortening of the hospitalization period. Further, risks such as damaging the vascular wall can be reduced. Further, transcatheter endovascular treatment can be applied to a wider range of cases to which the treatment has not been applicable so far, including cases of females and Asians having thin arteries.

The production of the woven fabric of the present embodiment is described below. In the step of providing the warp yarn constituting the woven fabric of the present embodiment, a required number of warp yarns are wound up on a warp beam using a warping machine, and the warp beam may be loaded in a loom. Alternatively, the warp yarns may be directly drawn onto a loom from yarn packages loaded in a creel.

The loom used for the production of the seamless tubular woven fabric of the present embodiment is not limited. It is preferred to use a shuttle loom in which the weft yarn is passed through by reciprocal movement of a shuttle, for production of the seamless woven fabric, and also for suppressing variation of the weaving density of the selvage portion of the woven fabric (the folded portion of the tubular woven fabric) to make the thickness of the woven fabric uniform. In cases where a shuttle loom is used, when there are two branch portions, the weaving may be carried out using three shuttles for the large-diameter portion, one branch portion, and the other branch portion, respectively. Alternatively, in cases where two shuttles are used, the weaving may be carried out using one shuttle for the large-diameter portion and one branch portion, and using the other shuttle for the other branch portion. By application of a constant tension during unwinding of the weft yarn from each shuttle, a high-quality tubular woven fabric having no wrinkles can be effectively woven. A structure using a plurality of springs or the like is preferably employed therefor. As described above, in cases where the woven fabric of the present embodiment is a straight fabric having no branch portion, weaving is possible by providing at least one shuttle containing the weft yarn, and the weft yarn can be continuous.

In weaving of a tubular woven fabric as in the present embodiment, a full-width temple may be used for the purpose of stabilizing the cloth fell, attaining a uniform thickness and diameter of the woven fabric, and suppressing yarn breakage and the like during processing. For the member of the full width temple in the portion in contact with the woven fabric, a material having a low friction coefficient is preferably selected. For the surface of the take-up roll, a tacky, non-slippery material having a smooth surface is preferably used. Regarding the structure of the full-width temple and the frictional coefficients of the members used, an appropriate design may be selected according to the monofilament fineness and the total fineness of each yarn used, and the weaving densities of the warp yarn and the weft yarn.

The weaving of the tubular seamless woven fabric requires a control of raising and lowering of the warp yarn. As the apparatus therefor, a Jacquard shedding apparatus, a dobby shedding apparatus, or the like may be employed. For easier formation of the woven texture of the branched portion, an electronic Jacquard machine is especially preferably used.

For changing the diameter of the tubular shape in the longitudinal direction, and/or for controlling the cover factor, the woven fabric may be prepared by performing reed beating using a reed in which the spaces between the dents vary in the vertical direction to thereby vertically change the reed beating position, or performing reed beating while vertically moving the weaving end.

After the weaving, scouring treatment for the purpose of removing the lubricant and the like, and heat setting for the purpose of shape stabilization are carried out. The temperature and the treatment time for the scouring, the temperature and the treatment time for the heat setting, and the tension in each process are not limited. For example, the graft may be treated under the following conditions: pre-heat setting at 150° C. for 30 minutes, scouring at 90° C. for 30 minutes, drying at 60° C. for 30 minutes, and final heat setting at 185° C. for 10 minutes. The treatment conditions may be appropriately determined according to the properties of the graft.

In cases where the woven fabric of the present embodiment is subjected to final heat setting, a metal jig for heat setting (heat setting bar) is preferably prepared as follows. A bar made of a metal such as aluminum or stainless steel having the diameter of the large-diameter portion, and a tapered metal bar having the diameter of the branch portion, are bound to each other such that no boundary appears. In cases where there is a shape change in the vicinity of the branched portion, the diameter of the bar is reduced correspondingly to shape change. Similarly, a cut setting bar having the diameter increased correspondingly to the shape change is preferably prepared. Preferably, in this case, from the viewpoint of workability, metal jigs for the large-diameter portion and the branch portion are separately produced such that they have structures enabling insertion of the metal jigs from the top and the bottom into the woven fabric to be subjected to the heat setting, and enabling their fixation in the woven fabric, to fix the woven fabric having the shape with the desired diameters without wrinkles.

The treated woven fabric is combined with a stent using a suture thread. The conditions for the joining of the woven fabric with the stent may be selected according to the shape of the stent. The needle used for the suture is not limited, and is preferably selected such that the water permeability after the needle puncture is not more than 500 ml/cm²/min. Subsequently, the stent graft obtained by the above method may be subjected to sterilization treatment. The conditions for the sterilization treatment are not limited, and may be selected taking into account the balance between the sterilization effect and, for example, the thermal shrinkage stress of the superfine polyester fiber after the treatment.

The present invention is concretely described below. However, the present invention is not limited to Examples. Common measurement values for physical properties were measured by the following methods.

### EXAMPLES

The present invention is concretely described below by way of Examples. However, the present invention is not limited to Examples. Common measurement values for physical properties were measured by the following methods.

### (1) Total Fineness and Monofilament Fineness

The total fineness (dtex) is measured for a 10-cm fiber bundle cut out from the large-diameter portion of the woven fabric. In the case of the warp yarn, the large-diameter portion is cut in the warp direction, and a warp yarn is pulled out from the cut end. In the case of the weft yarn, a spirally textured weft yarn is pulled out. The pulled-out yarn was dried to absolute dryness for 1 hour in an oven at 110° C. The yarn was then subjected to measurement of the weight using an analytical balance (SHIMADZU/AUW320), and the weight (g) was read to four decimal places, followed by calculation of the fineness (fineness based on corrected weight, F0) according to the following equation:

F0=1000×(m/L)×{(100+R0)/100}

{wherein F0 represents the fineness based on corrected weight (dtex); L represents the length of the sample (m); m represents the absolute dry mass of the sample; and R0 represents the official regain (%) defined in 3.1 of JIS-L-0105}.

The measurement was carried out 10 times for each case, and the average was rounded to the nearest integer.

The monofilament fineness (dtex) is the value obtained by dividing the total fineness calculated by the above method, by the number of monofilaments.

The total fineness of the branch portion can be measured in the same manner as the large-diameter portion.

In cases where the 10-cm fiber bundle cannot be sampled from the large-diameter portion or the branch portion, the longest fiber bundle that can be sampled from an area not overlapping with the tapered portion may be used to measure the total fineness by the same method.

### (2) Weaving Density

A square piece of at least 20 mm×20 mm was cut out from the woven fabric, and placed on a flat table. After removal of wrinkles, a pick counter (TEXTEST/FX3250) was perpendicularly placed with respect to the warp direction, and the warp density was measured. The displayed integer value was read. The measurement was carried out five times at different sites in the longitudinal direction of the woven fabric, and the average was rounded to one decimal place.

The weft density was measured in the same manner.

### (3) Cover Factor (CF)

Based on the total fineness determined in (1) and the weaving density determined in (2), CF was calculated according to the following equation:

CF=(√dw)×Mw+(√df)×Mf

{wherein dw represents the total fineness (dtex) of the warp yarn pulled out from the woven fabric; Mw represents the weaving density (yarns/2.54 cm) of the warp yarn; df represents the total fineness (dtex) of the weft yarn pulled out from the woven fabric; and Mf represents the weaving density (yarns/2.54 cm) of the weft yarn}.

The CF was rounded to the nearest integer. In the calculation of CF for a rib texture, since two warp yarns constituting the plain woven texture are combined to form one warp yarn having twice the fineness, the number of warp yarn was regarded as 1 while the fineness was doubled.

### (4) Twist Number

The twist number was measured for 10 yarns having a length of 100 mm pulled out from the large-diameter portion of the taper-shaped graft. The measurement was carried out for each of the warp yarn and the weft yarn.

In cases where the 100-mm fiber bundle cannot be sampled from the large-diameter portion, the longest fiber bundle that can be sampled from an area not overlapping with the tapered portion may be used to measure the total fineness by the same method.

### (5) Tensile Strength and Tensile Elongation

Regarding the tensile strength and the tensile elongation, a 300-mm yarn before weaving was collected according to JIS-L-1013, and measurement was carried out 10 times for each of the warp yarn and the weft yarn. For the measurement, Tensilon (EZ-LX), manufactured by Shimadzu Access Corporation, was used.

### (6) Burst Strength of Woven Fabric

According to ISO-7198, a burst strength test was carried out for the woven fabric. The base fabric was cut out as a piece of 40 mm×40 mm from each portion (large-diameter portion, tapered portion, or branch portion), and subjected to the measurement. In the sample collection from the tapered portion, the sample size of 40 mm×40 mm was secured by including the large-diameter portion and the branch portion as the top portion and the bottom portion, respectively, such that they have the same length. For example, in a case where the tapered portion has a length of 20 mm, a 10-mm area in the large-diameter portion and a 10-mm area in the branch portion were included as the top portion and the bottom portion, respectively. The measurement was carried out after placing the sample such that the tapered portion was positioned at the center. In cases where a sample having a sufficient size cannot be collected in the sample collection from the branch portion, the sample to be measured may be collected such that the sample can be set to the jig for the burst strength. In cases where the size was 30 mm×30 mm or the like, this fact may be recorded.

The measurement was carried out five times, and the average was rounded to the nearest integer.

### (7) Water Permeability of Woven Fabric

According to ISO-7198, the water permeability of the woven fabric was measured. The base fabric was cut out as a piece of 20×20 mm from each portion (large-diameter portion, tapered portion, or branch portion), and subjected to the measurement. The measurement was carried out five times, and the average was rounded to the nearest integer.

### (8) Water Permeabilities of Woven Fabric Before and After Needle Puncture

According to ISO-7198, the water permeability of the woven fabric was measured. The base fabric was cut out as a piece of 20 mm×20 mm from each portion (large-diameter portion, tapered portion, or branch portion), and a sewing-machine needle (DB×1 normal needle #11, manufactured by Organ Needle Co., Ltd.) was passed 10 times per 1 cm² in an arbitrary site, followed by performing the measurement. The measurement was carried out five times both before and after the needle puncture, and each average was rounded to the nearest integer.

### (9) Thickness of Woven Fabric

The base fabric was cut out as a piece of 20 mm×20 mm from each portion (large-diameter portion, tapered portion, or branch portion), and the measurement was carried out for arbitrary sites (n=10) using a thickness gauge according to ISO-7198 to read the thickness (μm). The resulting average was rounded to the nearest integer. FFD-10, manufactured by Ozaki Mfg. Co., Ltd., was used for the measurement.

(10) Insertability into Catheter

A woven fabric to which a stent was sutured was folded such that no unevenness occurred in the circumferential direction as seen from directly above, and whether or not it can be inserted into a catheter having a tubular inner diameter of 6 mm was evaluated. When the insertion was easy, a rating of “Easy” was given; when the insertion was hard, a rating of “Possible” was given; and, when the insertion was impossible, a rating of “Impossible” was given. Five samples were prepared for each condition, and evaluated.

### (11) Suture Strength

With reference to JIS-1096 (Method B of 8.21.1), test pieces were provided, and a test was carried out (n=5) until rupture occurs at the suture site of the woven fabric. The average of the maximum test force in the test was calculated.

Test pieces of 90 mm (length)×16 mm (width) whose warp direction, weft direction, or direction inclined at 45° to the warp direction is parallel to the tensile direction in the tensile test were provided. Each test piece was folded face-to-face in half so as to divide the length, and then cut along the fold. The resulting pieces were sewn together (lock stitch; 5 stitches/cm; sewing-machine needle, DB×1 normal needle #11 (manufactured by Organ Needle Co., Ltd.); sewing thread, polyester filament thread #50 (78 dtex×3; trade name, Ace Crown; manufactured by Onuki Limited)) along the line 10 mm distant from the cut end, and two back stitches were made at the beginning and ending of the sewing. Subsequently, the resulting test piece was drawn using a tensile tester with a grip distance of 30 mm at a tensile rate of 30 mm per minute. The maximum value of the force, at which the woven fabric was broken, was measured (n=5), and the average was calculated. In cases where collection of a sample having the length and the width is difficult, a sample with an arbitrary size may be collected as long as the measurement is possible therewith, and this fact may be recorded. For the measurement, Tensilon (EZ-LX), manufactured by Shimadzu Access Corporation, was used.

### (12) Sewing Thread Tensile Strength

According to ISO-7198, a rupture test of the woven fabric was carried out (n=5) for the sewing thread (polyester filament thread #50 (78 dtex×3; trade name, Ace Crown), manufactured by Onuki Limited) of the woven fabric. The average of the maximum test force in the test was calculated. For the measurement, Tensilon (EZ-LX), manufactured by Shimadzu Access Corporation, was used.

### (13) Bending Resistance

According to Method A of JIS L 1096 8.19.1 (45° cantilever method), a bending resistance test of the woven fabric was carried out (n=5), and the average was calculated.

### Example 1

A polyester fiber having a total fineness of 46 dtex/24F, a monofilament fineness of 1.9 dtex, a tensile strength of 4.7 cN/dtex, and a tensile elongation of 37% was twisted at a twist number of 440 T/m to provide the warp yarn, and a superfine polyester fiber having a total fineness of 26 dtex/140 F, a monofilament fineness of 0.19 dtex, a tensile strength of 4.1 cN/dtex, and a tensile elongation of 60% was twisted at a twist number of 90 T/m to provide the weft yarn. Using a shuttle loom provided with an electronic Jacquard shedding apparatus together with one shuttle, a straight tubular seamless woven fabric was prepared such that the entire woven fabric has a two-weft insertion double-woven structure. In the weaving, the number of warp yarns was 642; the reed width for the warp yarn was 54.2 mm; and the reed density was 14.8 dents/cm and 8 yarns/dent.

The woven fabric after the weaving was subjected to pre-heat setting, scouring, and heat setting under the following treatment conditions, to prepare a tubular woven fabric having a length of 302 mm and an inner diameter of 28 mm.

### (Pre-Heat Setting Conditions)

Pre-heat setting is carried out at 150° C. for 30 minutes.

### (Scouring Conditions)

With ultrapure water at 90° C., 30 minutes of washing is carried out twice by weak stirring.
Fixed-length drying is biaxially carried out at 60° C. for 30 minutes.

### (Final Heat Setting Conditions)

A stainless-steel bar having a diameter of 28 mm and a length of 400 mm is inserted into the scoured and dried woven fabric, and both ends of the woven fabric, having a length of 400 mm, are set and fixed using hose bands without causing wrinkles or looseness.
The stainless-steel bar to which the woven fabric is immobilized is placed in an incubator at 185° C. From the time point when the temperature in the incubator is controlled at 185° C., heat setting is carried out for 10 minutes.

### Comparative Example 1

A straight tubular seamless woven fabric was prepared in the same manner as in Example 1 except that a one-weft insertion woven structure was used, and that the weft density was adjusted. The length was 300 mm, and the inner diameter was 28 mm.

### Comparative Example 2

A straight tubular seamless woven fabric was prepared in the same manner as in Comparative Example 1 except that the same polyester fiber as the warp yarn, having a total fineness of 46 dtex/24 F and a monofilament fineness of 1.9 dtex, was used as the weft yarn, and that the weft density was adjusted. The length was 300 mm, and the inner diameter was 28 mm.

The water permeability, burst strength, tensile strength, suture strength, sewing thread tensile strength, bending resistance, weaving density, thickness, and cover factor of the straight tubular seamless woven fabrics prepared in Example 1, Comparative Example 1, and Comparative Example 2 are shown below in Table 1.

TABLE 1

| | | | | Comparative | Comparative |
|---|---|---|---|---|---|
| | | | Example 1 | Example 1 | Example 2 |
| Yarn | Warp | Yarn type | Regular | Regular | Regular |
| | yarn | | Polyester fiber | Polyester fiber | Polyester fiber |
| | | Total fineness | 46 | 46 | 46 |
| | | (dtex) | | | |
| | | Monofilament | 1.9 | 1.9 | 1.9 |
| | | fineness | | | |
| | | (dtex) | | | |
| | Weft | Yarn type | Superfine | Superfine | Regular |
| | yarn | | polyester fiber | polyester fiber | Polyester fiber |
| | | Total fineness | 26 | 26 | 46 |
| | | (dtex) | | | |
| | | Monofilament | 0.19 | 0.19 | 1.9 |
| | | fineness | | | |
| | | (dtex) | | | |

| | | Two-weft insertion | One-weft insertion | One-weft insertion |
|---|---|---|---|---|
| | | double-woven | double-woven | double-woven |
| | | structure | structure | structure |
| Woven texture | | (FIGS. 3, 4) | (FIGS. 1,2) | (FIGS. 1,2) |
| Woven | Warp density | 185 | 185 | 185 |
| fabric | (yarns/2.54 cm) | | | |
| | Weft density | 134 | 187 | 126 |
| | (yarns/2.54 cm) | | | |
| | Cover factor | 2221 | 2208 | 2109 |
| | Superfine fiber | 45 | 36 | 0 |
| | component ratio | | | |
| | (% by weight) | | | |
| Woven | Thickness (μm) | 88 | 76 | 80 |
| fabric | Burst strength (N) | 266 | 229 | 258 |
| evaluation | Water permeability | 85 | 131 | 534 |
| | (ml/cm²/min) | | | |
| | Water permeability | 89 | 139 | 575 |
| | after needle puncture | | | |
| | (ml/cm²/min) | | | |
| | Burst strength (N) | 9.3/9.3 | 10.0/4.5 | 9.7/9.2 |
| | Warp/Weft | | | |
| | Suture strength (N) | 17.3/15.0/11.5 | 16.4/10.5/7.7 | 15.4/13.2/12.0 |
| | Warp/45°/Weft | | | |
| | Sewing thread tensile | 92/78 | 89/63 | 91/75 |
| | strength (N) | | | |
| | Warp/Weft | | | |
| | Bending resistance (mm) | 61/32 | 64/26 | 63/41 |
| | Warp/Weft | | | |
| | Insertability into catheter | Easy | Easy | Easy |
| | (6-mm hole) | | | |

In Comparative Example 1, in which the superfine fiber is used as the weft yarn, and which uses a one-weft insertion woven structure, the suture strength at the angle perpendicular to the warp yarn was 16.4 N. However, the suture strengths at the angle perpendicular to the weft yarn and at the angle inclined at 45° from the warp direction were 7.7 N and 10.5 N, respectively, indicating decreased strength at the suture site. In contrast, Example 1, irrespective of the use of the superfine fiber as the weft yarn, showed an improved suture strength of not less than 11.5 N for any of the warp direction, 45° direction, and weft direction of the woven fabric because of the use of the two-weft insertion woven structure. Thus, the woven fabric of Example 1 has an increased strength at the suture site of the metal stent. Moreover, compared to Comparative Example 1, Example 1 showed an increase, from 26 mm to 32 mm, in the bending resistance in the weft direction, indicating improved shape retention.

In Comparative Example 2, in which, as the weft yarn, a regular yarn having a monofilament fineness of 1.9 dtex is used instead of the superfine fiber, and which uses a one-weft insertion woven structure, the water permeability was 534 ml/cm²/min, so that the property required for the graft for a stent graft is not satisfied.

### INDUSTRIAL APPLICABILITY

The woven fabric according to the present invention is a seamless tubular medical high density woven fabric which has the water permeability and the burst strength required for a material to be implanted in the body, which enables reduction of the diameter, which has an increased suture strength in a region of at least 10 mm in the longitudinal direction from one end of the fabric, and which is therefore capable of minimizing breakage at the suture site. Thus, the woven fabric can be suitably used as the graft for a stent graft.

1. A seamless tubular medical high density woven fabric, satisfying the following requirements (1) to (8):
(1) both warp yarn and weft yarn are synthetic multifilament fibers having a total fineness of not more than 60 dtex;
(2) the weft yarn has a monofilament fineness of not more than 0.5 dtex;
(3) the tubular woven fabric includes a two-weft insertion woven structure in a region of at least 10 mm in the longitudinal direction from one end of the tubular woven fabric;
(4) the woven fabric has a cover factor of 1600 to 2400; and
(5) the woven fabric has a thickness of not more than 110 μm.

2. The medical high density woven fabric according to claim 1, wherein the weft yarn is a synthetic polyester multifilament fiber having a monofilament fineness of not more than 0.2 dtex.
